(19) United States
(12) Patent Application Publication
(10) Pub. No.: US 2021/0098117 A1
(43) Pub. Date: Apr. 1, 2021

(54) HEALTHCARE PLATFORM TO CONNECT PATIENTS TO BETTER MEDICAL TREATMENTS

(71) Applicant: Patient Advisor LLC, Atlanta, GA (US)

(72) Inventor: Anthony Alex Hartley, Atlanta, GA (US)

(21) Appl. No.: 17/003,521

(22) Filed: Aug. 26, 2020

Related U.S. Application Data
(60) Provisional application No. 62/906,898, filed on Sep. 27, 2019.

(51) Int. Cl. G16H 40/20; G16H 50/70; G16H 10/20; G06Q 30/02; G06Q 30/00; G06Q 50/26; G16H 50/20; G16H 80/00; G06Q 10/10; G06Q 40/08; G06F 16/9535; G06F 16/9538; G06F 21/62; A61B 5/00

(52) U.S. Cl. CPC G16H 40/20; H04N 7/15; G16H 10/20; G06Q 30/0282; G06Q 30/018; G06Q 50/26; G16H 50/20; G16H 80/00; G06Q 30/0284; G06Q 10/1057; G06Q 40/08; G06F 16/9535; G06F 16/9538; G06F 21/6254; A61B 5/7465; G16H 50/70

(57) ABSTRACT

Disclosed are various embodiments for providing a healthcare application for the field of treatments using biological products such as stem cells. The healthcare application may connect users with providers that use biological products for treating medical conditions. Treatment options are generated for users who desire a treatment for a particular medical condition. The healthcare application may facilitate the user to schedule book the treatment, and gather healthcare outcomes and testimonials following the treatment.

### CROSS-REFERENCE TO RELATED APPLICATIONS

This application is a non-provisional application of, and claims priority to, co-pending U.S. Provisional application entitled “HEALTHCARE PLATFORM TO CONNECT PATIENTS TO BETTER MEDICAL TREATMENTS,” filed on Sep. 27, 2019, and assigned application No. 62/906,898, which is incorporated herein by reference in its entirety.

### BACKGROUND

The U.S. healthcare system is fragmented and by far the most expensive and difficult to handle as an individual consumer; no other country has more medical bankruptcies per year. Perhaps individuals visit WebMD® seeking knowledge and information for their medical needs. That website shows primarily medications and other products that have not reached a high-quality level of cure or improvement of health. In short, WebMD® and similar websites promote medications and other products whose efficacy is limited. The proliferation of self-service online resources results in fragmentation, which in turn requires healthcare consumers to rely on a plurality of disparate sources to navigate toward affordable healthcare. As can be seen, there is a need for a healthcare platform to connect patients to better medical treatments.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates a networked environment according to various example embodiments;

FIG. 2 illustrates a user interface rendered by a client in the networked environment of FIG. 1 according to various example embodiments;

FIG. 3 illustrates a user interface rendered by a client in the networked environment of FIG. 1 according to various example embodiments;

FIG. 4 illustrates a user interface rendered by a client in the networked environment of FIG. 1 according to various example embodiments;

FIG. 5 illustrates an example flowchart of certain functionality implemented by portions of a healthcare application executed in a computing environment in the networked environment of FIG. 1 according to various embodiments of the present disclosure; and

FIG. 6 is a schematic block diagram that illustrates an example computing environment and client(s) employed in the networked environment of FIG. 1 according to various embodiments.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions or positionings may be exaggerated to help visually convey certain principles. In the drawings, similar reference numerals between figures designate like or corresponding, but not necessarily the same, elements.

### DETAILED DESCRIPTION

As can be seen, there is a need for a healthcare platform to connect patients to better medical treatments. The present application relates to approaches for providing a healthcare application for the field of biological products, such as those products that can be derived from a patient or donor. Stem cell therapy and platelet-rich plasma (PRP) injections are both biological products that can assist in the regeneration of damaged tissue. Existing technologies may not be able to connect users with providers that use biological products for treating a particular medical condition, or connect users with providers through real-time consults with the providers. This disclosed approaches can vet or screen providers and biological products that are associated with beneficial or successful healthcare outcomes, generate treatment options for users who desire a treatment for a particular medical condition, facilitate the user to schedule an appointment or book the treatment, and/or provide mechanism(s) by which healthcare outcomes and testimonials can be gathered following treatment, among other things.

The innovative approaches described herein recognize that treatment options involving biological and other products can be generated that consider various aspects of medical conditions, providers, biological products, regulations, and healthcare benefits (e.g., benefits provided by or paid for by employers). The disclosed healthcare application can generate treatment options and track healthcare outcomes surpassing those of conventional healthcare websites or portals. Conventional healthcare websites and portals can face drawbacks or problems making them unsuitable or unable to connect patients to providers that use biological products for treatments. Embodiments disclosed herein can analyze treatment data to generate a criteria-based listing of providers (and/or biological products), generate treatment options associated with the providers, and provide an environment for a user to conduct real-time consultations with a subset of the providers, among other things. The approaches described herein can also relate to treatment options that can include pharmacological, surgical, laser therapy, gene therapy, immunotherapy, and therapeutic modalities that have yet to be determined, for which the healthcare application may be executed to connect users with options for treatments that are associated with beneficial or successful healthcare outcomes.

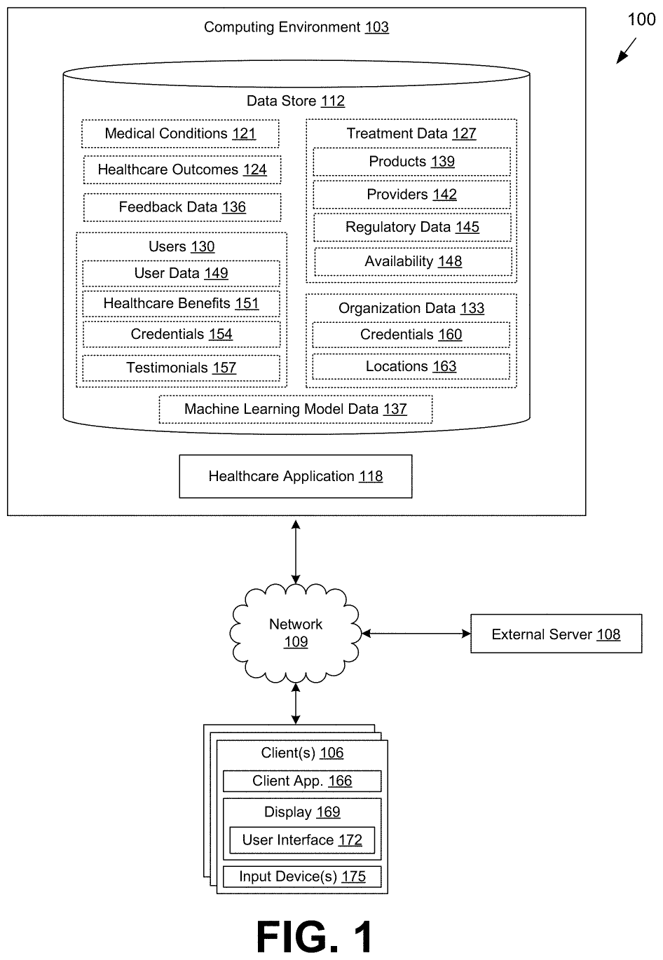

With reference to FIG. 1, shown is a networked environment 100 according to various embodiments. The networked environment 100 includes a computing environment 103, one or more clients 106, and external server 108 in data communication via a network 109. The network 109 includes, for example, the Internet, intranets, extranets, wide area networks (WANs), local area networks (LANs), wired networks, wireless networks, cable networks, satellite networks, or other suitable networks, etc., or any combination of two or more such networks. In some scenarios, the one or more clients 106 may include personal mobile clients. The external server 108 may be any computing device, computing environment, data provider, service provider, or computing resource provider, which may be provided by a third-party or by the provider of computing environment 103.

The computing environment 103 may comprise, for example, a server computer or any other system providing computing capability. Alternatively, the computing environment 103 may employ a plurality of computing devices that may be arranged, for example, in one or more server banks or computer banks or other arrangements. Such computing devices may be located in a single installation or may be distributed among many different geographical locations. For example, the computing environment 103 may include a plurality of computing devices that together may comprise a hosted computing resource, a grid computing resource, and/or any other distributed computing arrangement. In some cases, the computing environment 103 may correspond to an elastic computing resource where the allotted capacity of processing, network, storage, or other computing-related resources may vary over time.

Various applications and/or other functionality may be executed in the computing environment 103 according to various embodiments. Also, various data may be stored in a data store 112 that is accessible to the computing environment 103. The data store 112 may be representative of a plurality of data stores 112 as can be appreciated. The data stored in the data store 112, for example, is associated with the operation of the various applications and/or functional entities described below. The components executed on the computing environment 103, for example, include a healthcare application 118, and other applications, services, processes, systems, engines, or functionality not discussed in detail herein.

The healthcare application 118 may be executed to generate various network pages or user interfaces, including real-time consultation, to connect users such as patients to providers (e.g., hospitals, clinics, or clinicians) that use a biological product, other non-pharmaceutical product, or other product in a treatment for a medical condition. The present disclosure can involve biological products such as stem cells that are derived from a patient or donor. Product examples can also include System Biosciences® Exo-Flow™ Selective Exosome Capture, Theramex Ireland Ltd.'s Livogiva, EuroGenerics Holdings BV's Qutavina, and dupilumab (DUPIXENT®), or other products. Other example products include those associated with naturopathic, osteopathic, massage therapy, acupuncture, chelation therapy, barotherapy (e.g., oxygen), nutritional therapy, and other treatments as can be appreciated by one of skill in the art. The healthcare application 118 may be executed to generate data in the data store 112 that uniquely identifies products used in treatments in order to provide users with information about products, providers associated with the treatments, or information about healthcare outcomes involving the treatments.

The healthcare application 118 may be executed to vet or screen providers and biological products that are associated with beneficial or successful healthcare outcomes. The healthcare application 118 can vet providers by storing a taxonomy or listing in the data store 112 of providers that use biological products for treating a particular medical condition, and rank or sort the providers according to various criteria. The healthcare application 118 can also evaluate testimonials for providers, including by making sure that testimonials are legitimate. The healthcare application 118 may be executed to remove feedback or reviews that fail to mention a provider name, an outcome, a location, or that share more than a threshold amount of text with another review, and other such conditions that may indicate that a review is not legitimate or not helpful for vetting providers. The healthcare application 118 may index the testimonials by medical conditions involved.

In one embodiment, the healthcare application 118 may apply a machine learning algorithm to rank or sort the providers. The machine learning algorithm can be used to ascertain criteria for ranking or sorting the providers based upon an analysis of treatment data that is linked to providers, medical conditions, or healthcare outcomes. Providing user interfaces that allow users to rank or sort the providers by any and all possible criteria, or by criteria that is not necessarily correlated with positive healthcare outcomes, can result in systems that fail to connect users with appropriate providers. Searching a large number of providers that may or may not be associated with biological products can also present a technical challenge for conventional healthcare applications. Identifying the criteria for providers that have the most utility for particular biological products, medical conditions, or users can have a great deal of value. The machine learning algorithm can mine the treatment data to identify the criteria that is correlated with positive healthcare outcomes. To this end, the effectiveness of providers can be judged over time, and the criteria presented in one or more user interfaces to allow a user to rank or sort the providers by criteria which is correlated with medical conditions, healthcare outcomes, treatment data, or other data that may be relevant to the particular user.

The healthcare application 118 can vet products by searching the data store 112 for regulatory data, or data associated with using the products in treatments. The healthcare application 118 can also identify those products that are associated with beneficial or successful healthcare outcomes. The healthcare application 118 may be executed to aggregate data in (or obtain data from) the data store 112 that can assist the healthcare application 118 to generate one or more treatment options associated with providers using the biological product in the treatment for the medical condition. Aggregating data can include aggregating patient testimonials, searches, etc., without revealing any patient data, personally identifiable information, information whose disclosure may be prohibited under privacy law, or other types of information. In one approach, generating treatment options may be refined over time by tracking patient testimonials, searches, etc., and tracking healthcare outcomes over time to refine a machine learning model.

The healthcare application 118 may search the data store 112 or other sources for providers or biological products using criteria specified by a user. The healthcare application 118 may obtain a search request including search criteria or information describing a medical condition such as a symptom or a name of the medical condition. The healthcare application 118 may generate a result set of providers or biological products matching the search criteria. The search criteria can include a geographic location, an organization type, a credential, an accreditation, a healthcare benefit of the user, or other suitable criteria for searching providers or products.

The healthcare application 118 may generate a user interface comprising results from the search and cause the user interface to be rendered by one or more clients 106. For example, the healthcare application 118 may cause a client 106 to render the user interface by transmitting a web page to a web browser executed on client 106 or by transmitting information describing the search results to the client application, where the client application may be programmed to render a user interface including the search results.

The healthcare application 118 may be executed to provide and stream video and audio to users in a video conference using one or more clients 106. The healthcare application 118 may obtain a request from a user to initiate a video consultation with a subset of the providers, such as clinicians who perform a particular treatment. The request may include one or more criteria for the consult. For example, a user may request a consult with up to three providers, each being a board certified physician who has achieved expertise in a medical specialty or subspecialty, and accepting a “GreenPlus GreenSword” insurance company or a “Right to Shop” healthcare benefit. The healthcare application 118 may search the data store 112 for individuals presently available for a consult with the user.

The healthcare application 118 may initiate a video conference with the user and each of the one or more providers that meet the criteria specified by the user. A client 106 associated with each of the one or more providers may display a notification that the user wishes to consult with an option to accept or deny. The client 106 may display details of the case including payment estimates based at least in part on the healthcare benefits of the user and the type or severity of the medical condition. In some embodiments, the healthcare application 118 may be executed to extract parameters from the video conference to determine what words or content during the video conference may lead to a successful outcome (e.g., a booking for a treatment). The healthcare application 118 can provide words, content, or other feedback to providers during the consultation.

In one example, the healthcare application 118 looks at audio/video of the speech exchanges between providers and users to determine what are the ingredients to a successful consultation that makes the user go to that provider. The healthcare application 118 can overlay content onto the provider's display, e.g., telling the provider to “Be sure to mention [x] or [y] because 56% of patients have positive healthcare outcomes.”

The healthcare application 118 may be executed to facilitate a booking of one of the providers to perform the treatment on a day and time at a location. In some cases, the provider can be determined according to the healthcare benefits of the users. Healthcare benefits include, but are not limited to, those associated with insurance plans and insurance company. In some examples, the healthcare benefits are other benefits provided by an employer (including an employer associated with a municipal, state, or federal government), or those benefits established by legislation. In other examples, the provider is not determined according to the healthcare benefits of the users.

To the extent that locations of certain providers are outside of the country, etc., if the patient or treatment is not covered by a healthcare benefit or the cost according to the healthcare benefit is high, the healthcare application 118 may determine that the cost for treatment outside of the country is much less even after adding in the travel cost to get to that location outside of the country. The healthcare application 118 may be executed to compare costs to what a patient may be paying with or without insurance in the country, or to show an estimate of cost normalized by a measure of what the cost would be including travel coming from a location associated with the patient to the location outside the country. In one example, the healthcare application 118 adds an estimated cost (e.g., a cost obtained by querying a travel site) to a normalized providers cost and compares that to what out of pocket would be for a provider in the user's country.

Another example when a user might prefer to use the healthcare application 118 to book a treatment at a location outside his or her country is when a particular treatment doesn't have the right regulatory approval for a product to be used for the particular treatment in the user's country. The user could book the treatment at a clinic outside of the country and pay out of pocket. When the user returns home, he or she may have good healthcare outcomes resulting in not having to take any medicine. The healthcare application 118 can compare the out-of-pocket cost for treatment at the clinic outside of the country with the potential cost savings from the user not having to take any pharmaceuticals or medicine associated with the medical condition. It is also contemplated that the healthcare application 118 can offer a monetary payment to the user, for paying out of pocket and receiving out-of-network treatment, according to a healthcare benefit provided by the user's employer.

The healthcare application 118 may also be executed to obtain and store data in the data store 112 associated with the one of the providers performing the treatment. The healthcare application 118 may also generate a network page that renders or provides a series of prompts by which the user can build a testimonial. The testimonials can take many forms including text, audio, audio & video, and the like. The series of prompts can automate the interview process so that as the patient provides information, the healthcare application 118 renders additional questions or prompts to ask the patient about additional details of the treatment, the provider, or the product, if needed to create a testimonial having a threshold amount of detail. The healthcare application 118 can also create testimonials that are in compliance with Health Insurance Portability and Accountability Act (HIPAA) laws. For example, the healthcare application 118 can replace the patient's image with an avatar or some other representation, anonymize or remove identifying information, synthesize or alter the patient's voice, etc., so the testimonials do not qualify as Personally Identifiable Information (PII) that would be subject to HIPAA or other privacy laws.

The data stored in the data store 112 includes, for example, medical conditions 121, healthcare outcomes 124, treatment data 127, users 130, organization data 133, feedback data 136, machine learning model data 137, and potentially other data. The medical conditions 121 can include any data about medical conditions (e.g., sports injury, fibromyalgia, neuropathy, chronic pain, orthopedic condition, COVID-19, Lupus, Breast Cancer, Parkinson's, Alzheimer's, Sjogren's Syndrome, Dementia, Chronic obstructive pulmonary disease (COPD), and the like). The healthcare outcomes 124 can correspond to one or more measures that reflect the impact of a health care service, an intervention, or any use of a product in a treatment for one of the medical conditions 121.

The treatment data 127 may comprise various information about treatments for medical conditions 121, including products 139, providers 142, regulatory data 145, and availability 148. Products 139 may correspond to data about a biological product, other non-pharmaceutical product, or other product that can be used in a treatment for at least one of the medical conditions 121. In some cases, information about the products 139 may be organized within the data store 112 into a taxonomy to facilitate vetting or screening products 139 associated with healthcare outcomes 124.

The providers 142 can include hospitals, clinics, clinicians, or other providers who may use the products 139 in treatments for the medical conditions 121. The regulatory data 145 includes data corresponding to approvals or authorizations affecting the use of the products 139 by the providers 142, and other types of regulatory data. The healthcare application 118 can for example generate a network page that identifies a treatment as authorized, not authorized, or unregulated by a government agency based at least in part on the regulatory data 145. The availability 148 may include times during the week that a user associated with a provider 142 is available for a video conference. The availability may also be a flag indicating that a user is currently logged into the system awaiting a video conference. In some examples, the availability 148 is associated with availability of providers 142 to perform a treatment for one of the medical conditions 121.

The data stored in the data store 112 as the users 130 may include user data 149, healthcare benefits 151, credentials 154, and testimonials 157. Users 130 may include patients, insured individuals, payers, clinicians and supporting staff, such as nurses and physicians assistants, employers and employer representatives, or other users. The user data 149 may include profile information, such as name, address, and contact information. The user data 149 may also include patient records for one or more patients associated with one or more users, and other types of user data.

The healthcare benefits 151 can include data associated with coverage of particular products 139 and providers 142 under an insurance plan provided by an insurance company, or coverage according to a “Right to Shop” or other healthcare benefit provided by an employer. The healthcare benefits 151 may be imported from external server 108 in compliance with HIPAA and other privacy laws. The credentials 154 may include various credentials for a user such as college degrees, certificates, board certifications, expertise, training, awards, recognition, years of experience, feedback from other users including feedback for attitude, bedside manner, and various competences.

The testimonials 157 may include one or more digital content items comprising a testimonial or a link to a testimonial describing a user's experience associated with at least one of the providers 142 or products 139. Digital content items can include, but are not limited to, video files encoded according to a container format, links to videos on website such as YouTube® or Vimeo®, and numerous other types of digital content items that a user may view or interact with using the client 106.

The organization data 133 may include data linked to one or more users 130, providers 142, or may be separate from users 130 or providers 143. In some examples, users of the healthcare application 118 may include those users who are associated with providers 142, organizations, insurance companies, employers, and other users. The organization data 133 may include credentials 160 and locations 163 of the organization or one of the providers 142 associated with the organization. The credentials 160 may be the same as credentials 154 and may also include licensure, certification, accreditation (e.g., of the organization), recognition designation, certificates, privileges, or other credentials.

The feedback data 136 corresponds to various forms of user feedback about products 139, providers 142, or organizations. User feedback can include textual reviews, ratings, or other suitable feedback. For example, providers 142 may be given overall ratings by users (e.g., 3.5 out of 5 stars), and/or the ratings may be given across specific treatments or specific locations 163 where a treatment is performed. The machine learning model data 137 may correspond to data for one or more machine learning models used to ascertain criteria for ranking or sorting the providers 142, such as criteria based upon an analysis of treatment data 127 that is linked to providers 142, medical conditions 121, or healthcare outcomes 124. The machine learning algorithm can use the machine learning model data 137 to identify the criteria that is correlated with positive healthcare outcomes 124. The criteria can be presented in one or more user interfaces to allow a particular user 130 to rank or sort the providers 142 by criteria which is correlated with medical conditions 121, healthcare outcomes 124, treatment data 127, or other data that may be relevant to a particular user 130. The healthcare application 118 may be executed to track patient testimonials 157, searches, and healthcare outcomes 124, etc., over time to refine the machine learning algorithm and the machine learning model data 137.

The one or more clients 106 may be configured to execute various applications such as a client application 166 and/or other applications. The client application 166 may be executed in a client 106, for example, to access network content served up by the computing environment 103, thereby rendering a user interface 172 on the display 169. To this end, the client application 166 may comprise, for example, a browser, a dedicated application, etc., and the user interface 172 may comprise a network page, an application screen, etc. The client 106 may be configured to execute applications beyond the client application 166 such as, for example, email applications, word processors, spreadsheets, clinical applications, and/or other applications. The input devices 175 of the clients 106 may include a touch screen, a microphone, a camera, or one or more sensors. The client application 166 may render various user interfaces on the display 169 using extrinsic data to enhance the user experience. The user interfaces may be dynamically updated as a consultation progresses in the foreground or in the background.

In some embodiments, the healthcare application 118 ranks or sorts providers 142 according to healthcare outcomes 124, savings (or potential savings) to users 130, risk to the users 130, products 139 used, or medical conditions 121 treated. Some medical conditions 121, e.g., knee injury, and some products 139, e.g., stem cells, may be more common than others. For ranking providers 142 who are associated with medical conditions 121 that are relatively uncommon or which the healthcare outcomes 124 for the medical conditions 121 are not associated with a threshold number of treatments or procedures, the healthcare application 118 can aggregate data from other medical conditions 121 that are associated with the providers 142.

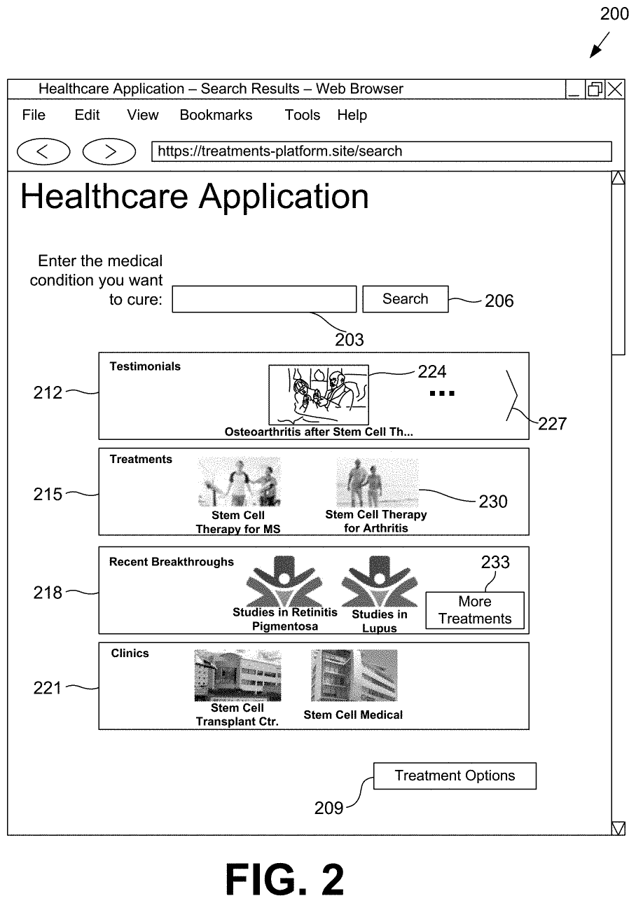

With reference to FIG. 2, shown is an illustration of a search user interface 200 rendered on a display 169 (FIG. 1) of a client 106 (FIG. 1) in the networked environment of FIG. 1. In some examples, the search user interface 200 may be used to search for information about medical conditions 121 (FIG. 1) or any of the data stored in the data store 112 (FIG. 1). The search user interface 200 may include a search box 203, a search button 206, and a treatment options button 209. The search user interface 200 may include one or more groupings of information related to the medical condition, such as testimonials 212, treatments 215, recent breakthroughs 218, and clinics 221 as depicted in FIG. 2. The testimonials 212, treatments 215, recent breakthroughs 218, and clinics 221 may be generated by the healthcare application 118 (FIG. 1), for example, when a user selects the search button 206.

In some examples, the search user interface 200 may present information tailored to a specific condition of the medical conditions 121 (FIG. 1). If a user has a certain condition, the user can specify a certain condition the user is looking for in the search box 203 and sort through testimonials 212, etc. In some other examples, the search user interface 200 may present a combination of therapies and conditions that are treated regardless of what the user may or may not enter in the search box 203.

Testimonials 212 can include one or more content items 224, which can be arranged on a carousel and advanced via the element 227. When selected, the content item 224 allows streaming of a video item stored or linked as one of the testimonials 157 (FIG. 1).

Treatments 215 can include one or more content items 230 for presenting a network page containing information associated with treatment data 127 (FIG. 1), including information about one or more products 139 used to treat medical conditions 121. Recent breakthroughs 218 can include information about studies or other information available to the healthcare application 118 that is relevant to one of the medical conditions 121. For example, the recent breakthroughs 218 can provide information about research and educational videos so the patient can understand the field of medicine related to treating one or more of the medical conditions 121. The clinics 221 can include clickable images, icons, names, descriptions, and/or other suitable user interface elements associated with organizations identified in the organization data 133 (FIG. 1) or providers 142 (FIG. 1).

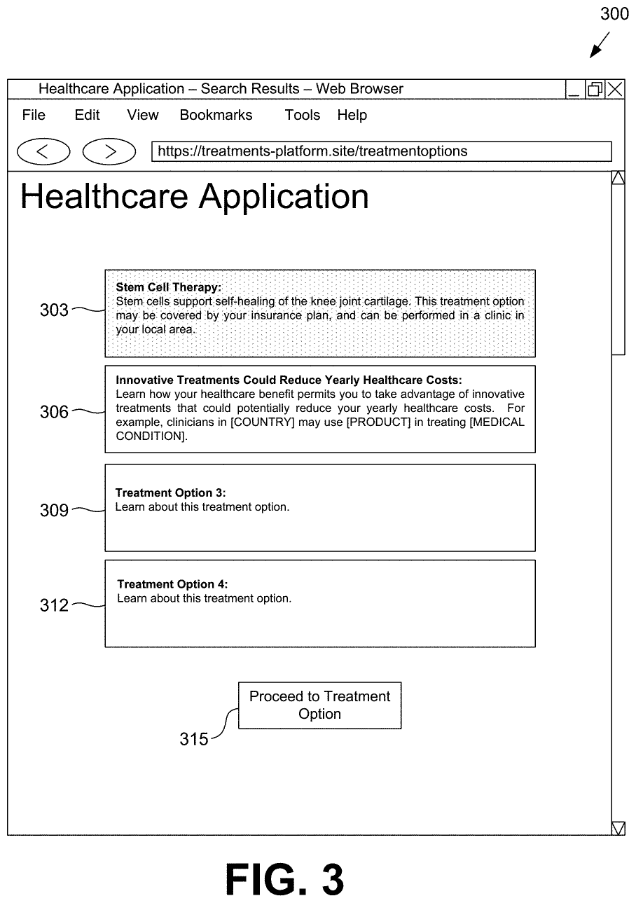

The treatment options button 209 can be used to generate a network page that presents various treatment options related to text entered in the search box 203 or related to the testimonials 212, treatments 215, recent breakthroughs 218, or clinics 221. The healthcare application 118 may for example generate a plurality of treatment options associated with providers 142 using at least one of the products 139 in the treatment for the medical condition. A user that desires treatment can select the treatment options button 209, and proceed towards treatment using an embodiment according to FIG. 3.

With reference to FIG. 3, shown is an illustration of a treatment options user interface 300 rendered on a display 169 (FIG. 1) of a client 106 (FIG. 1) in the networked environment of FIG. 1. The treatment options user interface 300 may include one or more treatment options 303, 306, 309, or 312, such as those associated with a particular one of the providers 142 (FIG. 1), products 139 (FIG. 1), or healthcare benefits 151 (FIG. 1). In some embodiments, a user may select a treatment option by clicking the treatment option, such as clicking within the box of treatment option 303. In other embodiments, clicking the treatment option highlights the treatment option, such as by greying the box of the item, and a user must select a user interface element, such as the Proceed to Treatment Option button 315 to advance to the treatment option.

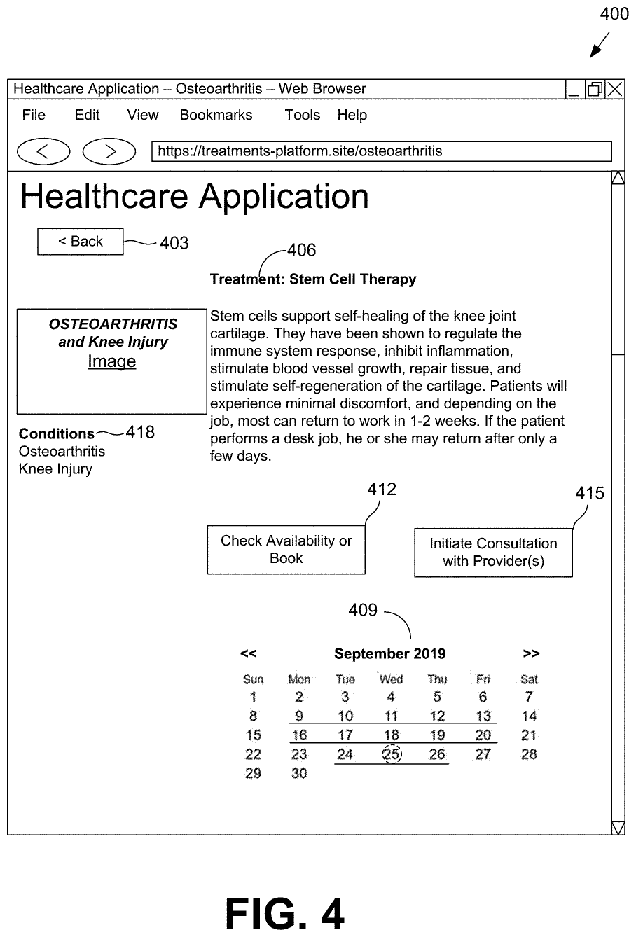

The treatment option may include a check box to indicate which one or more of the treatment options 303, 306, 309, or 312 is currently selected. When a treatment option is proceeded to, a user may advance to view information about the treatment option, initiate a consultation with a provider, or book the treatment option. For example, a user that selects the treatment option 303 for stem cell therapy, may proceed to stem cell therapy treatment using an embodiment according to FIG. 4.

With reference to FIG. 4, shown is an illustration of a treatment option user interface 400 rendered on a display 169 (FIG. 1) of a client 106 (FIG. 1) in the networked environment of FIG. 1. The treatment option user interface 400 may include a back button 403, description of the treatment option 406, a calendar 409, a check availability or book button 412, an initiate video consult button 415, and a list 418 associated with the medical conditions 121 (FIG. 1) related to the treatment option.

The back button 403 may return the user to a previous screen. The description of the treatment option 406 may include any information about the treatment option or why the treatment option was generated for the user.

The calendar 409 may display a calendar showing days or periods that are potentially available for treatments. The days or periods can correspond to the availability 148 (FIG. 1). The user can interact with the calendar (e.g., to select one of the days). The check availability or book button 412 may facilitate a booking of one of the providers 142 (FIG. 1) to perform the treatment on a day and time, such as a day selected in the calendar 409.

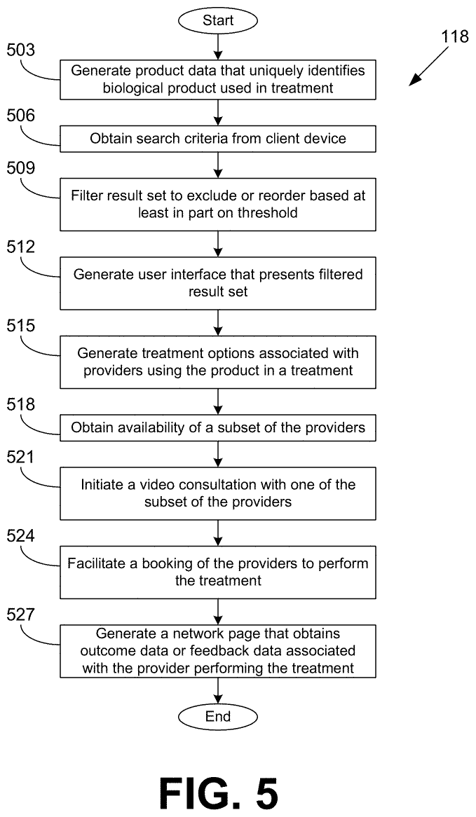

Referring next to FIG. 5, shown is a flowchart that provides one example of the operation of a portion of the healthcare application 118 according to various embodiments. It is understood that the flowchart of FIG. 5 provides merely an example of the many different types of functional arrangements that may be employed to implement the operation of the portion of the healthcare application 118 as described herein. As an alternative, the flowchart of FIG. 5 may be viewed as depicting an example of elements of a method implemented in the computing environment 103 (FIG. 1) according to one or more embodiments.

Beginning with box 503, the healthcare application 118 generates data about products 139 (FIG. 1) that uniquely identifies a biological product used in a treatment for one or more medical conditions 121 (FIG. 1). For example, the healthcare application 118 may assign a unique identifier to each of the products 139. In some embodiments, the healthcare application 118 obtains a listing of providers 142 (FIG. 1) of the treatment.

At box 506, the healthcare application 118 obtains a search request from a client 106 (FIG. 1) including search criteria and information describing a medical condition of a user, which can be one of the medical conditions 121 (FIG. 1). For example, a user may enter a keyword into the search box 203 of the search user interface 200 shown in FIG. 2. Information describing the medical condition can include a symptom, a name of the medical condition, or other information associated with the medical condition. The search criteria can include a geographic location, an organization type, a credential, an accreditation, a healthcare benefit of the user, or other suitable criteria for searching the data store 112.

At box 509, the healthcare application 118 generates a result set of providers 142 that match the search criteria by executing a search on the data store 112. The healthcare application 118 may generate a filtered result set of providers 142 by filtering the result set to exclude or reorder providers 142 based at least in part on relevance criteria. In one example, providers 142 who are not associated with at least a threshold of successful outcomes identified in the healthcare outcomes 124 may be removed or excluded from the result set.

In one example, providers 142 with items of feedback data 136 having a review title exceeding a maximum length may be excluded, as such a title may reflect a poor quality review that is not associated with testimonials 157 or healthcare outcomes 124. In another example, the result set may be filtered to exclude low-rated providers 142 or providers 142 that have not been used at least a minimum number of times.

At box 512, the healthcare application 118 generates a user interface that presents the filtered result. Data encoding the user interface is sent to the client 106 via the network 109 (FIG. 1) for rendering upon the display 169 (FIG. 1).

At box 515, the healthcare application 118 generate a plurality of treatment options associated with at least a portion of the providers 142 (FIG. 1) using one of the products 139 (FIG. 1) in a treatment for the medical condition of the user. The healthcare application 118 can receive or determine a selection of one or more of the providers 142 to treat the condition. For example, the user may select one or more radio buttons corresponding to the selection of at least a portion of the providers 142. The selection may be returned from the client 106 to the healthcare application 118 via the network 109.

At box 518, the healthcare application 118 obtains an availability of the at least a portion of the providers 142. At box 521, the healthcare application 118 may involve obtaining a request from a user who desires to consult with the providers 142. For example, The request may include one or more criteria for the providers 142 that the user wishes to consult or conference. The healthcare application 118 can initiate a video conference with the user and one or more providers 142 meeting the criteria from the user request. The healthcare application 118 may send a request to a client 106 associated with each of the one or more providers to enter into a video chat via a camera connected to or part of the client 106. Upon obtaining an indication of an answer from one or more providers, the healthcare application 118 may connect each answered provider via a video conference.

At box 524, the healthcare application 118 facilitates, based on the availability, a booking of one of the providers 142 to perform the treatment on a day and time at a location. The location can for example be one of the locations 163 that are associated with organization data 133 or a location of an organization where the treatment can be performed.

At box 527, the healthcare application 118 generates a network page that obtains healthcare outcomes 124 or feedback data 136 associated with the one of the providers performing the treatment. The healthcare application 118 can also provide a series of prompts by which the user can build one of the testimonials 157. The healthcare application 118 can generate a network page that renders or provides a series of prompts by which the user 130 can build one of the testimonials 157. The series of prompts can automate the interview process so that as the user 130 provides information, the healthcare application 118 renders additional questions or prompts to ask the user 130 about details of the treatment, the provider 142, or the product 139. For example, the network page can prompt the user 130 to provide data (text, audio, audio & video, and the like). The user 130 can be prompted to provide data associated with his or her medical condition 121 (or other data associated with why the user 130 sought treatment), and a length of stay for the user 130 at a location 163 of an organization associated with the provider 142. In some examples, the user 130 can be prompted to indicate one of the products 139 used in treatment, and to briefly describe an experience following the treatment that the healthcare application 118 can associated with the healthcare outcomes 124. Thereafter, the portion of the healthcare application 118 can end.

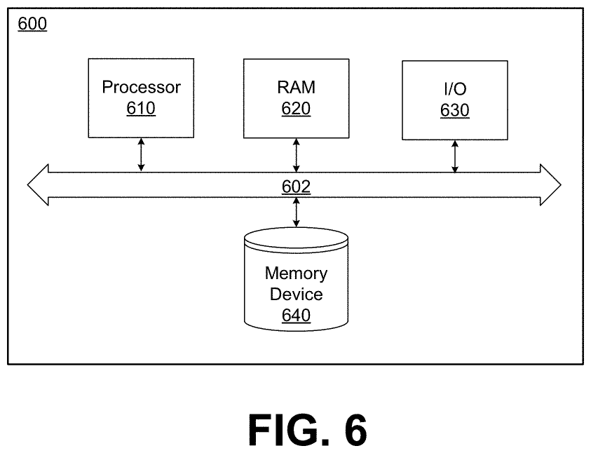

With reference to FIG. 6, shown is a schematic block diagram 600 of the computing environment 103, client 106, and/or external server 108 according to an embodiment of the present disclosure. The computing environment 103, client(s) 106, and external server 108 include one or more computing devices. Each computing device includes at least one processor circuit, for example, having a processor 610 and a memory 640, both of which are coupled to a local interface 602. To this end, each computing device may comprise, for example, at least one server computer or like device. The local interface 602 may comprise, for example, a data bus with an accompanying address/control bus or other bus structure as can be appreciated.

Stored in the memory 640 are both data and several components that are executable by the processor 610. In particular, stored in the memory 640 and executable by the processor 610 are healthcare application 118 and client application 166, and potentially other applications. Also stored in the memory 640 may be a data store 112 and other data. In addition, an operating system may be stored in the memory 640 and executable by the processor 610.

It is understood that there may be other applications that are stored in the memory 640 and are executable by the processor 610 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 640 and are executable by the processor 610. In this respect, the term “executable” means a program file that is in a form that can ultimately be run by the processor 610. Examples of executable programs may be, for example, a compiled program that can be translated into machine code in a format that can be loaded into a random access portion of the memory 640 and run by the processor 610, source code that may be expressed in proper format such as object code that is capable of being loaded into a random access portion of the memory 640 and executed by the processor 610, or source code that may be interpreted by another executable program to generate instructions in a random access portion of the memory 640 to be executed by the processor 610, etc. An executable program may be stored in any portion or component of the memory 640 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 640 is defined herein as including both volatile and nonvolatile memory and data storage components. Volatile components are those that do not retain data values upon loss of power. Nonvolatile components are those that retain data upon a loss of power. Thus, the memory 640 may comprise, for example, random access memory (RAM) (e.g., RAM 620), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 610 may represent multiple processors 610 and/or multiple processor cores and the memory 640 may represent multiple memories 640 that operate in parallel processing circuits, respectively. In such a case, the local interface 602 may be an appropriate network that facilitates communication between any two of the multiple processors 610, between any processor 610 and any of the memories 640, or between any two of the memories 640, etc. The local interface 602 may comprise additional systems designed to coordinate this communication, including, for example, performing load balancing. The processor 610 may be of electrical or of some other available construction.

Although healthcare application 118 and client application 166, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 5 shows the functionality and operation of an implementation of portions of the healthcare application 118. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 610 in a computer system or other system. The machine code may be converted from the source code, etc. If embodied in hardware, each block may represent a circuit or a number of interconnected circuits to implement the specified logical function(s).

Although the flowchart of FIG. 5 shows a specific order of execution, it is understood that the order of execution may differ from that which is depicted. For example, the order of execution of two or more blocks may be scrambled relative to the order shown. Also, two or more blocks shown in succession in FIG. 5 may be executed concurrently or with partial concurrence. Further, in some embodiments, one or more of the blocks shown in FIG. 5 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including healthcare application 118 and client application 166, that comprises software or code can be embodied in any non-transitory computer-readable medium for use by or in connection with an instruction execution system such as, for example, a processor 610 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including healthcare application 118 and client application 166, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device, or in multiple computing devices in the same computing environment 103. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A non-transitory computer-readable medium embodying a program that, when executed by at least one computing device being configured to at least:
generate product data that uniquely identifies a biological product used in a treatment for a medical condition;
obtain a listing of a plurality of providers of the treatment associated with the biological product;
generate a plurality of treatment options associated with at least a portion of the providers using the biological product in the treatment for the medical condition;
facilitate a booking of one of the providers of the at least a portion of the providers to perform the treatment on a day and time at a location; and
generate a network page that obtains healthcare outcome data and renders a series of prompts for a user to provide feedback data comprising a testimonial associated with the one of the providers performing the treatment.

2. The non-transitory computer-readable medium of claim 1, wherein the at least one computing device is further configured to at least:
obtain a search request from a client device including search criteria and information describing the medical condition of the user; and
generate a result set of providers matching the search criteria.

3. The non-transitory computer-readable medium of claim 2, wherein the at least one computing device is further configured to at least:
generate a filtered result set of providers based at least in part on removing or excluding from the result set of providers any providers who are not associated with at least a threshold of successful outcomes; and
generate a user interface that presents the filtered result set of providers.

4. The non-transitory computer-readable medium of claim 2, wherein the search criteria comprises at least one of: a geographic location, an organization type, a credential, an accreditation, or a healthcare benefit of the user.

5. The non-transitory computer-readable medium of claim 1, wherein the computing device is further configured to filter or rank the plurality of treatment options based at least in part on a healthcare benefit of the user.

6. A system, comprising:
a data store; and
at least one computing device in communication with the data store, the at least one computing device being configured to at least:
generate product data that uniquely identifies a biological product used in a treatment for a medical condition;
obtain a listing of a plurality of providers of the treatment associated with the biological product;
obtain a search request from a client device including search criteria and information describing the medical condition of a user;
generate a result set of providers matching the search criteria;
generate a plurality of treatment options associated with at least a portion of the providers using the biological product in the treatment for the medical condition;
obtain an availability of the at least a portion of the providers;
facilitate, based at least in part on the availability, a booking of one of the providers of the at least a portion of the providers to perform the treatment on a day and time at a location;
generate a network page that obtains at least one of: healthcare outcome data or feedback data associated with the one of the providers performing the treatment; and
in an instance in which the network page obtains the feedback data, provide a series of prompts by which the user can build a testimonial.

7. The system of claim 6, wherein the information describing the medical condition of the user comprises at least one of: a symptom or a name of the medical condition.

8. The system of claim 6, wherein the at least one computing device is further configured to at least:
obtain regulatory data associated with the at least the portion of the providers using the biological product in the treatment for the medical condition; and
generate a network page that identifies the treatment as authorized, not authorized, or unregulated by a government agency based at least in part on the regulatory data.

9. A method, comprising:
generating, via at least one computing device, product data that uniquely identifies a biological product used in a treatment for a medical condition;
obtaining, via the at least one computing device, a listing of a plurality of providers of the treatment associated with the biological product;
obtaining, via the at least one computing device, a search request from a client device including search criteria and information describing the medical condition of a user;
generating, via the at least one computing device, a result set of providers matching the search criteria;
generating, via the at least one computing device, a plurality of treatment options associated with at least a portion of the providers using the biological product in the treatment for the medical condition;
obtaining, via the at least one computing device, a request to initiate a video consultation with a subset of the providers of the result set of providers;
initiating, via the at least one computing device, the video consultation with at least one of the subset of the providers; and
facilitating, via the at least one computing device, a booking of the at least one of the subset of the providers.

10. The method of claim 9, further comprising removing or excluding from the result set of providers any providers who are not associated with at least a threshold of successful outcomes.

11. The method of claim 9, further comprising removing or excluding from the result set of providers any providers who are not associated with at least a threshold of testimonials.

12. The method of claim 9, wherein generating at least one of the treatment options comprises generating, via the at least one computing device, an estimate of cost normalized by a measure of what the cost would be including travel.

13. The method of claim 12, further comprising generating, via the at least one computing device, a page that presents the cost compared to a potential cost savings from the user not having to take any pharmaceuticals associated with the medical condition.

14. The method of claim 9, further comprising overlaying, via the at least one computing device, words or content onto a display of one of the providers during the video consultation.

15. The method of claim 9, wherein the search criteria comprises a healthcare benefit of the user comprising a Right to Shop healthcare benefit.

16. The method of claim 9, wherein the information describing the medical condition of the user comprises at least one of: a symptom or a name of the medical condition.

17. The method of claim 9, further comprising selecting, via the at least one computing device, the subset of the providers based at least in part on the providers meeting at least one criteria comprising a credential or a location specified in the request to initiate the video consultation.

18. The method of claim 9, further comprising generating, via the at least one computing device, a series of prompts by which the user can build a testimonial.

19. The method of claim 18, further comprising replacing, via the at least one computing device, a user image embodied in a plurality of frames of the testimonial with an avatar or other representation.

20. The method of claim 18, further comprising removing, via the at least one computing device, personally identifying information from metadata of the testimonial.
